(19) United States
(12) Patent Application Publication
(10) Pub. No.: US 2008/0286876 A1
(43) Pub. Date: Nov. 20, 2008

(54) GENES ASSOCIATED WITH ALZHEIMER'S DISEASE - Hltdip

(76) Inventor: Chissoe Stephanie, Durham, NC (US)

Correspondence Address: GLAXOSMITHKLINE;Corporate Intellectual Property, Five Moore Drive, P.O. Box 13398, Research Triangle Park, NC, 27709-3398, US

(21) Appl. No.: 12/117,845

(22) Filed: May 9, 2008

Related U.S. Application Data
(60) Provisional application No. 60/917,767, filed on May 14, 2007.

(51) Int. Cl. G01N 33/00

(52) U.S. Cl. 436/86

(57) ABSTRACT

A method of screening a small molecule compound for use in treating Alzheimers Disease, comprising screening a test compound against a target or targets selected from the gene products encoded by a group of specified genes, where activity against said target indicates the test compound has potential use in treating Alzheimers Disease.

### CROSS-REFERENCE TO RELATED APPLICATIONS

This application claims priority to U.S. Provisional Application No. 60/917,767 filed May 14, 2007.

### FIELD OF THE INVENTION

The present invention relates to identification of genes that are associated with Alzheimer's Disease (AD) and to screening methods to identify chemical compounds that act on those targets for the treatment of Alzheimer's Disease or its associated pathologies.

### BACKGROUND OF THE INVENTION

The purpose of the present study was to identify genes coding for tractable targets that are associated with Alzheimer's Disease, to develop screening methods to identify compounds that act upon such targets, and to develop such compounds as medicines to treat Alzheimer's Disease and its associated pathologies.

Alzheimer's disease (AD), the most prevalent form of dementia, is a complex neurodegenerative disorder that affects over 10% of individuals aged 65 years and older. While mutations in amyloid precursor protein (APP), presenilin-1 (PS-1), and presenilin-2 (PS-1) genes are associated with rare, autosomal dominant inherited forms of familial AD, there is also evidence for a significant genetic contribution to sporadic late-onset AD. Family history of AD in a first degree relative is associated with increased risk of AD in case-control studies. Twin studies estimate the pairwise concordance rate of twins as high as 83% for monozygotic twins and 46% for dizygotic twins (Bergem et al., 1997; Gatz et al., 1997; Gatz et al. 2006). The discovery of the association between the apolipoprotein E (APOE) ε4 allele and AD provided evidence for the role of specific genetic risk factors in late-onset AD (Corder e al, 1993; Saunders et al., 1993). Multiple epidemiological studies confirm APOE as a major susceptibility gene, also influencing age of onset and pathological expression of the disease.

Identification of genes associated with common forms of AD provides an opportunity to develop novel therapeutic targets, or identify potential genetic factors influencing phenotypic expression of disease or responses to treatment. The identification of these genes, even of small and combined effects, will be of considerable therapeutic importance, and will help establish a complete picture of the pathophysiology of the disease. Ultimately, a better understanding of the underlying pathophysiology of the disease would permit more rational drug development and provide a valuable strategy for the development of innovative drugs to treat the disease and reduce both its social and economical burden (Roses et al, 2005).

### SUMMARY OF THE INVENTION

A first aspect of the present invention is a method for screening small molecule compounds for use in treating Alzheimer's Disease (AD), by screening a test compound against a target selected from the group consisting of ACHE, ARS2, APOC1, APOE, TOMM40, CCBP2, CCDC21, CCL26, CSNK2A1, GPRC5A, MMP10, MMP1, C190RF22, CFD, ELA2, PRTN3, THRAP5, ROR2, SCAMP5, SCG2, SLC25A4, TXNDC14, ABCD2, BUB1, CCL11, CPB2, FGR, IKBKB, IL2RB, JAK1, PPARBP, RIPK4, SLC1A2, APBB3, SLC35A4, SRA1, YES1, ALK, C5, CHRNB4, CLCN7, IL16, KCNQ5, PAK2, and PPAT. Activity against said target indicates the test compound has potential use in treating Alzheimer's Disease.

### DETAILED DESCRIPTION

The present inventors tested genes that encode for potential tractable targets to identify genes that are associated with the occurrence of AD and to provide methods for screening to identify compounds with potential therapeutic effects in AD. An assessment of AD data was carried out with a full data set of all 1270 Caucasian cases and 1215 Caucasian controls collected from Canada and the UK. Allelic and genotypic frequencies for the 11,045 Single Nucleotide Polymorphisms (SNPs) in 2,033 loci were contrasted between the cases and controls. In addition, locus-based permutation analyses were performed to account for the variable number of SNPs per locus. On the basis of these analyses, 14 loci encompassing 22 genes were identified as being significantly associated with Alzheimer's Disease: ACHE (genes ACHE, ARS2), APOE APOC1 (genes TOMM40, APOE, APOC1), CCBP2, CCDC21, CCL26, CSNK2A1, GPRC5A, MMP10_MMP1 (genes MMP10, MMP1), PRTN3_THRAP5 (genes C190RF22, CFD, ELA2, PRTN3, THRAP5), ROR2, SCAMP5, SCG2, SLC25A4, TXNDC14. These loci all have a locus-based permutation P≦0.005 in the full data set. Likewise, an additional 13 loci (15 genes) showed statistical significance in the full data set with a permutation P>0.005 but ≦0.01.

These are ABCD2, BUB1, CCL11, CPB2, FGR, IKBKB, IL2RB, JAK1, PPARBP, RIPK4, SLC1A2, SRA1 (genes APBB3, SLC35A4, SRA1), and YES1. An assessment adjusting for APOE status and collection site revealed 8 more statistically significant genes with observed P≦0.001: ALK, C5, CHRNB4, CLCN7, IL16, KCNQ5, PAK2, and PPAT.

As used herein, a ‘tractable target’ or ‘druggable target’ is a biological molecule that is known to be responsive to manipulation by small molecule chemical compounds, e.g., can be activated or inhibited by small molecule chemical compounds. Classes of ‘tractable targets’ include, but are not limited to, 7-transmembrane receptors (7TM receptors), ion channels, nuclear receptors, kinases, proteases and integrins.

### EXAMPLE 1

### Subjects and Methods

### Sample Set

This is a case/control study design and subjects were recruited through two collections. The Canadian collection includes case-control subjects from a multi-centre study in Canada that recruited 874 Caucasian Alzheimer's disease patients and 847 age, sex and ethnicity matched controls. Inclusion criteria required that AD patients satisfied NINCDS-ADRDA (McKhann et al., 1984) and DSM-IV (American Psychiatric Institute, 1994) criteria for AD, had an Mini-Mental State Exam (MMSE)≦26 (Folstein et al, 1975), a Global Deterioration Scale (GDS) of 3-7 (ranging from mild to very severe cognitive decline) (Reisberg et al. 1982), and had clinical data supporting the diagnosis of AD. Ethnically matched controls were recruited from non-biological relatives, friends, or spouses of the cases. Controls had no history of memory impairment, MMSE≧27, Mattis Dementia Rating Scale (DRS)≧136 (Mattis et al. 1976; Schmidt et al. 1994), Clock Test (11:10) without error, and no impairment of the 7 instrumental ADL questions from the Duke Older American Resources and Services Procedures (OARS) (Fillenbaum et al. 1988) caused by cognitive decline. Cases or controls were excluded if they were currently in a major depressive episode, psychosis, or acute manic or depressive episode of bipolar disorder. A second collection from Cardiff UK was comprised of 453 cases and 477 controls. The case subjects collected in Cardiff also met the ADRDA/NINCDS criteria for diagnosis of probable Alzheimer's disease and the control subjects are matched for age, sex and ethnicity. All subjects gave informed consent for the use of their DNA in this study.

### Target Genes

Relatively few human proteins, currently approximately a hundred in total, are considered to be suitable targets for effective small molecule medicines. It was considered reasonable to include all the members of these families for which a sequence was available. At the time, some of the genes were not exemplified in the public domain and were discovered through the analysis of expressed sequence tags or genomic sequence using a combination of sequence analysis. In addition, genes were selected because they were the targets of effective drugs even though they were not part of large protein families. Finally, disease expertise was employed to select genes whose involvement in AD was either proven or suspected. Genes were named accordingly to NCBI ENTREZ Gene.

### SNP Identification

The genes were automatically assembled and annotated with a region of the gene designated as 5′ and 3′, intron and exon. SNPs were mapped using BLAST to the manually curated genomic sequences. The SNPs were selected up to 10 kb from the start and stop sites of the transcripts with an average intermarker distance of 30 Kb. SNPs with a minor allele frequency (MAF)>5% were selected, but all coding SNPs known at the time were included irrespective of MAF. Approximately 10% of genes had fewer than 6 SNPs and these were subjected to SNP discovery using 24 primer pairs per gene to amplify 12 DNA samples selected from Coriell Cell Repository of female CEPH cell-line samples. (CEPH refers to the Centre d'Etude du Polymorphisme Humain, which collected Northern European DNA samples.) For all of the discovered SNPs a minor allele frequency was determined using the FAST (Flow Accelerated SNP Typing) (Taylor et al, 2001) technology using multiplex PCR coupled with Single Base Chain Extension (SBCE) and Amplifluor genotyping. A marker selection algorithm was used to remove highly correlated SNPs to reduce the genotyping requirement while maintaining the genetic information content throughout the regions (Meng et al, 2003).

### Sample Preparation and Genotyping

DNA was isolated from whole blood using a basic salting-out procedure. Samples were arrayed and normalized in water to a standard concentration of 5 ng/ul. Twenty nanogram aliquots of the DNA samples were arrayed into 96-well PCR plates. For purposes of quality control, 3.4% of the samples were duplicated on the plates and two negative template control wells received water. The samples were dried and the plates were stored at −20° C. until use. Genotyping was performed by a modification of the single base chain extension (SBCE) assay previously described (Taylor et al. 2001). Assays were designed by a GlaxoSmithKline in-house primer design program and then grouped into multiplexes of 50 reactions for PCR and SBCE. Following genotyping, the data was scored using a modification of Spotfire Decision Site Version 7.0 Genotypes passed quality control if: a) duplicate comparisons were concordant, b) negative template controls did not generate genotypes and c) more than 80% of the samples had valid genotypes. Genotypes for assays passing quality control tests were exported to an analysis database.

Genotyping was conducted in two waves over the course of recruitment. Wave1 consisted of the approximately first 500 cases and 500 controls collected. Wave2 consisted of the remaining cases and controls collected after Wave1. Over time the gene and SNP list expanded such that the Wave2 subjects were genotyped on a larger number of genes and SNPs than the earlier recruited Wave1 subjects. The Wave1 genotyping was conducted in 2004 after the first 984 Canadian subjects were recruited (484 cases, 500 controls). These subjects were genotyped for 5,652 SNPs in 1,651 loci. The Wave2 genotyping was conducted in 2006 on an additional 737 Canadian subjects (390 cases, 347 controls) and 930 Cardiff subjects (453 cases, 477 controls). The Wave2 subjects were genotyped for 9,992 SNPs in 2,001 loci. The 1^{st} and 2^{nd} wave subjects were genotyped on 5,134 SNPs in common.

SNPs typed in Wave1 only: 518

SNPs typed in Wave2 only: 4,858

SNPs typed in both waves: 5,134

SNPs typed in Wave1 and/or Wave2: 10,510

### Data Handling

The GSK database of record for analysis-ready data is called SubjectLand. This database contains all genotypes, phenotypes (i.e. clinical data), and pedigree information, where applicable, on all subjects used in the analysis of data for these studies. SubjectLand does not maintain information regarding DNA samples, but is closely integrated with the sample tracking system to maintain the connection between subjects and their samples and phenotypic data at all times. All subjects gave informed consent for the use of their DNA and phenotypic data in this study. The analytical tools used in the analysis process described below interface directly with subject data in SubjectLand. This interface also archives the files used in analysis as well as the results.

### Analysis

Only subjects with a subject type (SBTY) of case or control were analyzed. Subjects with a SBTY of affected family member or other SBTY values were excluded from analysis. Subjects were also excluded if he/she, either parent, or more than one grandparent were non-Caucasian as indicated by self-report. In addition, subjects were excluded if their putative gender was inconsistent with SNP genotypes on the X chromosome. Finally, subjects that genotyped on fewer than 75% of the SNPs in a given genotyping experiment were excluded from analysis.

Each marker was examined for Hardy-Weinberg equilibrium (HWE) and minor allele frequency. Markers which were monomorphic in cases and controls or that were out of HWE in controls with a p-value≦0.001 were excluded from analysis. Also excluded were markers that mapped to multiple locations in the genome.

Genotypic and allelic associations test were then performed, followed by identification of the risk allele and risk genotype using chi-square tests. An odds ratio and confidence interval of greater than 95% was calculated for the risk allele and risk genotype. Next, population stratification was evaluated by determining if the number of allelic and genotypic tests observed to be significant at a given threshold was inflated with respect to what would be expected under the null hypothesis of no association. In addition, linkage disequilibrium (LD) was examined to measure the association between alleles at different loci (Weir, 1996, pp. 109-110). Lastly, a locus-based permutation assessment was conducted to account for the variable number of SNPs per locus and yield a single permutation p-value per locus for the full analysis data set of cases and controls. Statistically significant loci were identified as those passing locus-based permutation thresholds. The empirical permutation p-value from the full data set was required to fall at or below 0.01 to be considered significantly associated with Alzheimer's Disease. This set of loci was then further categorized such that loci with a permutation p-value less than 0.005 were considered as having the strongest evidence for association to AD.

APOE is a known AD risk factor and APOE4 status was determined by APOE genotypes at two SNPs: Cys112Arg (RS429358) and Cys158Arg (RS7412). APOE4 status was defined as negative if a subject carried 0 copies of the ε4 allele and positive if a subject carried 1 or 2 copies of the ε4 allele (with the exception that APOE4 status was not defined for subjects with an ε2/ε4 genotype as the ε2 allele is protective and tends to cancel out the effect of the ε4 allele). Three analyses were conducted to account for the effect of APOE on AD. A logistic regression analysis was conducted adjusting for APOE4 status as well as for collection site (Canada or UK). Two case control association analyses were conducted in the two separate subsets stratified by APOE4 status: an APOE4 positive subset and an APOE4 negative set.

APOE SNPs: RS429358 and RS7412:

| | | Corresponding |
|---|---|---|
| SNP name | SNP allele | Amino Acid |
| RS429358 | T | Cys112 |
| RS429358 | C | Arg112 |
| RS7412 | T | Cys158 |
| RS7412 | C | Arg158 |

APOE-2, APOE-3 and APOE-4 allele status corresponds to
RS429358 and RS7412 alleles:

| | SNP alleles defining APOE 2/3/4 | Corresponding |
|---|---|---|
| Allele Name | allele status | Amino Acids |
| ApoE2 | RS429358 allele T and RS7412 allele T | Cys112-Cys158 |
| ApoE3 | RS429358 allele T and RS7412 allele C | Cys112-Arg158 |
| ApoE4 | RS429358 allele C and RS7412 allele C | Arg112-Arg158 |

Correspondence between genotype combinations and
derived APOE genotype.

| RS429358 | RS7412 | APOE | APOE4 Allele | |
|---|---|---|---|---|
| Genotype | Genotype | Genotype | Copies | APOE4 status |
| CC | CC | ε4/ε4 | 2 | Positive |
| CT | CC | ε 3/ε4 | 1 | Positive |
| CT | CT | ε 2/ε4 | 1 | Omitted |
| TT | CC | ε 3/ε3 | 0 | Negative |
| TT | CT | ε 2/ε3 | 0 | Negative |
| TT | TT | ε 2/ε2 | 0 | Negative |
| CC | CT | ?, ?* | ?* | Not seen |
| CC | TT | ?, ?* | ?* | Not Seen |
| CT | TT | ?, ?* | ?* | Not Seen |

*This combination of genotypes has not been observed

### Hardy Weinberg Equilibrium

Hardy Weinberg equilibrium (HWE) is a measure of the association between two alleles at an individual locus. A bi-allelic marker is in HWE if the genotype frequencies are p2, 2pq and q2 for the genotypes 1, 1; 1, 2; and 2, 2 where p and q are the frequencies of the 1 and 2 alleles, respectively. The departure from HWE was tested using a Chi square test, by testing the difference between the expected (calculated from the allele frequencies) and observed genotype frequencies. A HWE permutation test was performed when the HWE chi-square p-value<0.05 and when at least one genotype cell had an expected count <5 (Zaykin et al, 1995). When these conditions exist, the HWE chi-square test may not be valid and a permutation test to assess departure from HWE is warranted. Markers failing HWE at p≦0.001 in controls were removed from the analysis marker cluster used in association analyses. HWE failure may indicate a non-robust assay.

### Minor Allele Frequency

For minor allele frequency, markers which were monomorphic were removed from the analysis marker cluster used in association analyses.

### Allelic and Genotypic Test of Association

Testing for association in the study data was carried out using the ‘PROC FREQ’ fast Fisher's exact test (FET) procedure in the statistical software package SASv8.2. An exact test is warranted in situations when asymptotic assumptions are not met such as when the sample size is not large or when the distribution is sparse or skewed. Such situations occur for SNPs with rare minor allele frequencies where the numbers of expected cases and/or controls for the rare homozygote are fewer than five. Under these conditions, the asymptotic results many not be valid and the asymptotic p-value may differ substantially from the exact p-value. The classic Fisher's Exact Test computes exact p-values by enumerating all tables as extreme as, or more extreme than, that observed. This direct enumeration approach is very time-consuming and only feasible for small problems. The fast Fisher's Exact test computes exact p-values for general R×C tables using the network algorithm developed by Mehta and Patel (1983). The network algorithm provides substantial advantage over direct enumeration and is rapid and accurate.

Tables I and II show the structure of the genotype and allele contingency tables, respectively.

TABLE I
Generic disease status by genotype contingency table.
Disease Status

| | | Case | Control | Total |
|---|---|---|---|---|
| Genotype | AA | n11 | n12 | n1. |
| | Aa | n21 | n22 | n2. |
| | aa | n31 | n32 | n3. |

| | | | |
|---|---|---|---|
| Total | n.1 | n.2 | N |

TABLE II
Generic disease status by allele contingency table.
Disease Status

| | | Case | Control | Total |
|---|---|---|---|---|
| Allele | A | 2n11 + n21 | 2n12 + n22 | 2n1. + n2. |
| | a | 2n31 + n21 | 2n32 + n22 | 2n3. + n2. |

| | | | |
|---|---|---|---|
| Total | 2n.1 | 2n.2 | 2N |

### Risk Allele and Risk Genotype

The “risk allele” refers to the allele that appeared more frequently in cases than controls. The “risk genotype” was determined after identifying the genotype that had the largest chi-square value when compared against the other 2 genotypes combined in the genotypic association test. For example, if a SNP had genotypes AA, AG and GG, 3 chi-square tests were performed contrasting cases and controls: 1) AA vs AG+GG, 2) AG vs AA+GG and 3) GG vs AA+AG. An odds ratio was then calculated for the test with the largest chi-square statistic. If the odds ratio was >1, this genotype was reported as the risk genotype. If the odds ratio was <1, then 1) the risk genotype was reported as “!” (“!” means “not”) this genotype and 2) a new odds ratio was calculated as the inverse of the original odds ratio. This new odds ratio was reported.

### Odds Ratios and Confidence Intervals

An odds ratio was constructed for the risk allele and risk genotype.

Odds ratio (OR)=(n11*n22)/(n12*n21)

where

n11=cases with risk genotype

n21=cases without risk genotype

n12=controls with risk genotype

n22=controls without risk genotype

In order to avoid division or multiplication by zero, 0.5 was added to each cell in the contingency table (as recommended in “Statistical Methods for Rates and Proportions” by Fleiss, Ch 5.3 p. 64)

OR*exp(−z\sqrt{j}),OR*exp(z\sqrt{v})

A 95% confidence interval for the odds ratio was also calculated as follows: where

z=97.5th percentile of the standard normal distribution

v=[1/(n11)]+[1/(n12)]+[1/(n21)]+[1/(n22)].

### Evaluation of Population Stratification

In this assessment, cases and control frequencies were compared across a subset of relatively independent markers (markers in low LD) selected from the set of all markers analyzed. Since the vast majority of genes on the gene list are not associated with a specific disease, this constitutes a null data set. If the cases and controls are from the same underlying population, the expectation is to see 5% of the tests significant at the 5% level, 1% significant at the 1% level, etc. If, on the other hand, the cases and controls are from different populations, (for example, cases from Finland and controls from Japan), there would be an inflation in the proportion of tests significant across thresholds due to genetic differences between the two populations that are unrelated to disease. Inflation in the number of observed significant tests over a range of cut-points suggests that the case and control groups are not well matched. Consequently, the inflated number of positive tests may be due to population stratification rather than to association between the associated SNPs and disease.

The probability of ≧m observed number of significant tests out of n total tests at a cut-point p was calculated using the binomial probability as implemented in SAS. PROBNML (p,n,m) computes the probability that an observation from a binomial (n,p) distribution will be less than or equal to m.

### Linkage Disequilibrium

The LD between two markers is given by DAB=pAB−pApB, where pA is the allele frequency of A allele of the first marker, pB is the allele frequency of B allele of the second marker, and pAB is the joint frequency of alleles A and B on the same haplotype. LD tends to decline with distance between markers and generally exists for markers that are less than 100 kb apart

The SAS procedure PROC CORR was used to calculate r using the Pearson product-moment correlation. To determine whether significant LD existed between a pair of markers we made use of the fact that nr2 has an approximate chi square distribution with 1 df for biallelic markers. The significance level of pairwise LD was computed in SAS.

### Permutation Assessment

The analysis of the observed un-permuted data led to a set of observed p-values for each locus. We defined min [obs(p)] as the minimum p-value derived from all tests of all SNPs within the locus for a given data set. The objective of this permutation test was to determine the significance of this minimum p-value in context of the number of SNPs analyzed number of tests conducted and the correlation between SNPs within each locus. The permutation process accounted for the multiple SNPs and tests conducted within a particular locus but it did not account for the total number of loci being analyzed.

Due to computational limitations, only those loci with a min [obs (p)] less than a threshold of 0.05 were assessed for significance using a permutation process. A maximum number of permutations, N, was conducted per locus (N=50,000 for full set; see below). However, this maximum number did not need to be conducted for every locus. For many loci far fewer permutations were sufficient to show that a locus was not significant at the threshold of interest and the permutation process for that locus was terminated early.

The following process was followed. For each permutation, affection status was shuffled among the cases and controls, maintaining the overall number of cases and number of controls in the observed data. The genetic data for each subject were not altered. For each permutation, all the SNPs within a locus were analyzed using allelic and genotypic association tests (same methods as employed with true, observed data). The p-value for the most significant test, min [sim (p)] was captured for each permutation. The permutations were repeated up to N times such that up to N min [sim (p)]'s were captured. Once the permutations were completed, the min [obs (p)] for each locus was compared against the distribution of min [sim (p)]. The proportion of min [sim (p)] that was less than the min [obs (p)] gave the empirical permutation p-value for that locus. This p-value was labelled perm (p).

The maximum number of iterations needed to accurately assess the permutation p-value depended on the threshold set for declaring significance. For example, in assessing permutation p-values below 0.05, 5000 permutations gave a 95% confidence interval (CI) of 0.044 to 0.056. This was not considered to be a tight enough estimate of the true permutation p-value. By assessing 50,000 permutations the 95% CI was narrowed considerably, to 0.48 to 0.52. The CIs for a range of permutation p-values and numbers of permutations are presented below.

| permP | 5000 CI | 10000 CI | 50000 CI |
|---|---|---|---|
| 0.05 | (0.044, 0.056) | (0.0457, 0.0543) | (0.048, 0.052) |
| 0.01 | (0.0072, 0.0128) | (0.008, 0.012) | (0.0091, 0.011) |
| 0.005 | (0.003, 0.008) | (0.0036, 0.0064) | (0.0044, 0.0056) |

Based on the above CI estimates, loci in the full data set with an obs (p)≦0.05 were assessed with a maximum of 50,000 permutations.

### EXAMPLE 2

### Results

Out of the 2,651 subjects genotyped, 166 subjects were excluded from the study based on sample set quality control (QC) measures; 70 for ethnicity (the Canadian collection included 70 subjects of Ashkenazi Jewish or Native American descent), 27 for gender inconsistency, 10 who did not meet inclusion criteria for subject type, and 59 that were genotyped on fewer than 75% of the SNPs. Key demographic characteristics of the 2,485 analyzed subjects (1,270 cases and 1,215 controls) are detailed in Table 1. APOE4 status for analyzable subjects is provided in Table 2. The proportion of ε4 positive subjects by case/control status is similar in both collections. The majority of cases, 61%, are APOE4 positive (have at least 1 copy of the ε4 allele) compared to only 23% of the controls.

During SNP marker quality control, a total of 629 SNPs were excluded: 118 SNPs were excluded due to deviation from Hardy-Weinberg Equilibrium (HWE); 439 SNPs were excluded because SNPs were monomorphic in cases and controls; 6 SNPs were excluded as they returned genotypes on <75% of samples, and 66 SNPs were excluded due to mapping issues. As a result, 9,881 out of the 10,510 SNPs genotyped were analyzable for association with AD. These 9,881 SNPs map to 2,033 loci. In addition, 1,164 coding SNPs that mapped to the 2,033 loci were also analyzed; these additional SNPs were genotyped on the full set of subjects (wave1 and wave2). In total, 11,045 SNPs (9,881+1,164) in 2,033 loci were analyzed of which 10,937 had a locus assignment and 108 did not. Of the 2,033 loci analyzed, 1,960 were autosomal and 73 were X-linked. The mean number of SNPs per locus was 5.4 with a range of 1-185 SNPs per locus. See Table 3 for a summary SNP coverage of loci.

Detailed summaries of genotype counts across all markers and subjects analysed are given in Table 4 and Table 5. The apparent bimodal distribution seen in the tables reflect the staged genotyping process and the evolution of the gene list over time.

After locus-based permutation analysis, 14 loci (22 genes) were identified as having the strongest statistical evidence for genetic associated with AD (Table 6). The set of loci reached a locus-based permutation P-value of <=0.005 in the overall data set of all 1,270 cases and 1,215 controls. The 13 loci (15 genes) in Table 7 are the next best in terms of statistical evidence. These have a locus-based permutation P-value between 0.005 and 0.01. The two collections were also separately assessed. The only locus passing a Bonferroni threshold in each of these collections was the APOE locus for rs429358, which encodes the Cys112Arg polymorphism and is one of the two polymorphisms which determines APOE alleles ε2, ε3 and ε4 (Canadian collection observed p=1.94E−62, Cardiff collection observed p=1.32E−27). Genes significant in analyses adjusting for APOE effects are listed in Table 8.

The number of tests significant across various thresholds was not inflated beyond what is expected by chance (Table 9-11) indicating that there was no significant evidence of population stratification between collections or between cases and controls.

### Discussion

ACHE, ARS2, APOC1, APOE, TOMM40, CCBP2, CCDC21, CCL26, CSNK2A1, GPRC5A, MMP10, MMP1, C190RF22, CFD, ELA2, PRTN3, THRAP5, ROR2, SCAMP5, SCG2, SLC25A4, TXNDC14, ABCD2, BUB1, CCL11, CPB2, FGR, IKBKB, IL2RB, JAK1, PPARBP, RIPK4, SLC1A2, APBB3, SLC35A4, SRA1, and YES1 passed statistically significant locus-based permutation thresholds in the full data set. These genes have the strongest statistical evidence for association with Alzheimer's Disease. Eight additional genes were identified as associated (observed p<0.001) in the APOE analyses: ALK, C5, CHRNB4, CLCN7, IL16, KCNQ5, PAK2, and PPAT.

Further, there was no evidence of population stratification based on the distribution of results. However, it is possible that some of the associations are false positives. Statistical association between a polymorphic marker and disease may occur for several reasons. The marker may be a mutation that influences disease susceptibility directly or may be correlated with a mutation that influences disease susceptibility because the marker and disease susceptibility mutation are physically close to one another. Spurious association may result from issues such as confounding or bias although the study design attempts to remove or minimize these factors. The association between a marker and disease may also be due to chance.

The locus-wise type 1 error is the locus-based permutation p-value threshold used to identify the genes of interest. It also provides the false positive rate associated with each locus. Out of 2,033 loci examined, an average of 10.2±3.2 would be expected to have a permutation p≦0.005 while 20.3±4.5 would be expected to have a permutation p≦0.01.

TABLE 1
Collections analysed

| | Canada | | Cardiff UK | |
|---|---|---|---|---|
| | Cases | Controls | Cases | Controls |
| Number of | 821 | 785 | 449 | 430 |
| Subjects | | | | |
| Gender (M:F) | 341:480 | 280:505 | 118:331 | 106:324 |
| (% Males) | (41.5%) | (35.7%) | (26.3%) | (24.7%) |
| Age At Interview¹ | 78.0 ± 8.6 | 73.4 ± 8.0 | 81.4 ± 6.6 | 76.2 ± 6.1 |
| | (43, 100) | (48, 94) | (63, 97) | (65, 95) |
| Age At Onset¹ | 72.2 ± 8.5 | | 75.7 ± 6.8 | |
| | (40, 97) | | (60, 92) | |
| Age At Diagnosis¹ | 75.1 ± 8.3 | | N/A² | |
| | (40, 97) | | | |

¹Mean ± Std Dev in years (min, max)
²Age at Diagnosis not available in Cardiff collection

TABLE 2
APOE4 status in Cases and Controls

| | Canada | | Cardiff UK | | Full Set | |
|---|---|---|---|---|---|---|
| | Cases | Controls | Cases | Controls | Cases | Controls |
| Number of Subjects | 784 | 756 | 432 | 422 | 1216 | 1178 |
| with ε4 status¹ | | | | | | |
| Number of ε4 | 487 (62%) | 172 (23%) | 249 (58%) | 93 (22%) | 736 (61%) | 265 (22.5%) |
| Positive Subjects | | | | | | |
| (%)² | | | | | | |
| Number of ε4 | 297 (38%) | 584 (77%) | 183 (42%) | 329 (78%) | 480 (39%) | 913 (77.5%) |
| Negative Subjects | | | | | | |
| (%)² | | | | | | |

¹The number of subjects with ε4 status is less than the total number in Table 1 because some subjects could not have ε4 status assigned. These subjects either lacked a genotype at 1 or both of the 2 APOE SNPs used to determine the ε2, ε3 or ε4 alleles (rs7412, rs429358) or had APOE genotype ε2/ε4
²Percentages are within cases or within controls. For example, among the 784 Canadian cases with ε4 status, 62% were ε4 positive (487/784) and 38% were ε4 negative (297/784).

TABLE 3
SNP coverage of loci analyzed

| | | | | | 6-9 | 10+ | |
|---|---|---|---|---|---|---|---|
| | 1 SNP | 2 SNPs | 3 SNPS | 4-5 SNPs | SNPs | SNPs | Total |
| No. loci | 219 | 420 | 372 | 478 | 315 | 229 | 2,033 |

TABLE 4
Summary of subject genotype counts across SNPs

| Numbers of | Number |
|---|---|
| subjects genotyped | of markers |
| 2350-2485 | 3,854 |
| 2250-2349 | 2,046 |
| 2000-2249 | 260 |
| 1550-2000 | 0 |
| 1400-1549 | 1,055 |
| 1250-1399 | 3,396 |
| 750-1249 | 434 |

TABLE 5
Summary of SNP genotype counts across subjects

| Numbers of | Number |
|---|---|
| SNPs genotyped | of subjects |
| 10001-11,045 | 1,325 |
| 9001-10000 | 67 |
| 8001-9000 | 7 |
| 7001-8000 | 0 |
| 6001-7000 | 899 |
| <=6000 | 187 |

TABLE 6
Loci with a Permutation P ≦ 0.005¹

| | | Permutation | | | |
|---|---|---|---|---|---|
| | Chr | P- | | | |
| Locus² | Location | value | Gene³ | Target Class | Gene Description |
| ACHE | 7q22 | 0.00311 | ACHE | LIPASE_ESTERASE | Acetylcholinesterase (YT blood group) |
| | | | ARS2 | Unclassified | Arsenate resistance protein ARS2 |
| APOE_APOC1 | 19q13.2 | 2.0E−05 | APOE | OTHER_TARGETS | Apolipoprotein E |
| | | | APOC1 | Unclassified | Apolipoprotein C-I |
| | | | TOMM40 | Unclassified | Translocase of outer mitochondrial membrane |
| | | | | | 40 homolog (yeast) |
| CCBP2 | 3p21.3 | 0.0006 | CCBP2 | 7TM | Chemokine binding protein 2 |
| CCDC21 | 1p36.11 | 0.0023 | CCDC21 | Unclassified | Hypothetical protein DKFZp434L0117 |
| CCL26 | 7q11.23 | 0.0023 | CCL26 | 7TM_LIGAND | Chemokine (C-C motif) ligand 26 |
| CSNK2A1 | 20p13 | 0.0041 | CSNK2A1 | KINASE | Casein kinase 2, alpha 1 polypeptide |
| GPRC5A | 12p13-p12.3 | 0.0020 | GPRC5A | 7TM | Retinoic acid induced 3 |
| MMP10_MMP1 | 11q22.3 | 0.0009 | MMP1 | PROTEASE | Matrix metallopropeinase 1 (interstitial collagenase) |
| | | | MMP10 | PROTEASE | Matrix metalloproteinase 10 (stromelysin 2) |
| PRTN3_THRAP5 | 19p13.3 | 0.0034 | C19ORF22 | Unclassified | Hypothetical protein BC012775 |
| | | | CFD | PROTEASE | D component of complement (adipsin) |
| | | | ELA2 | PROTEASE | Elastase 2, neutrophil |
| | | | PRTN3 | PROTEASE | Proteinase 3 (serine proteinase, neutrophil, Wegener |
| | | | | | granulomatosis autoantigen) |
| | | | THRAP5 | NR COFACTOR | Thyroid hormone receptor-associated protein, 95-kD subunit |
| ROR2 | 9q22 | 0.0011 | ROR2 | KINASE | Receptor tyrosine kinase-like orphan receptor 2 |
| SCAMP5 | 15q23 | 0.0049 | SCAMP5 | Unclassified | Secretory carrier membrane protein 5 |
| SCG2 | 2q35-q36 | 0.0044 | SCG2 | OTHER_TARGETS | Secretogranin II (chromogranin C) |
| SLC25A4 | 4q35 | 0.0044 | SLC25A4 | TRANSPORTER | Solute carrier family 25 (mitochondrial carrier, adenine |
| | | | | | nucleotide translocator), member 4 |
| TXNDC14 | 11cen-q22.3 | 0.0020 | TXNDC14 | Unclassified | CGI-31 protein |

¹The set of LOCI that have reached a locus-based permutation P-value of <=0.005 in the full data set of all 1,270 cases and 1,215 controls.
²Locus is a label used to assign a 1:1 relationship between a SNP and a unique part of the genome. In most instances the locus and gene are one in the same. However, in gene rich parts of the genome (where SNPs map to multiple genes), a locus may include several genes.
³In gene rich parts of the genome, some loci may have SNPs which map to several genes or have overlapping genes. Disease association may be to any of these genes.

TABLE 7
Loci with a Permutation P between 0.005 and 0.01¹

| | | Permutation | | | |
|---|---|---|---|---|---|
| | Chr | P- | | | |
| Locus² | Location | value | Gene³ | Target Class | Gene Description |
| ABCD2 | 12q11-q12 | 0.0074 | ABCD2 | TRANSPORTER | ATP-binding cassette, sub-family D (ALD), member 2 |
| BUB1 | 2q14 | 0.0010 | BUB1 | KINASE | BUB1 budding uninhibited by benzimidazoles 1 homolog (yeast |
| CCL11 | 17q21.1-q21.2 | 0.0063 | CCL11 | 7TM_LIGAND | Chemokine (C-C motif) ligand 11 |
| CPB2 | 13q14.11 | 0.0080 | CPB2 | PROTEASE | Carboxypeptidase B2 (plasma, carboxypeptidase U) |
| FGR | 1p36.2-p36.1 | 0.0073 | FGR | KINASE | Gardner-Rasheed feline sarcoma viral (v-fgr) oncogene homolog |
| IKBKB | 8p11.2 | 0.0074 | IKBKB | KINASE | Inhibitor of kappa light polypeptide gene enhancer in B-cells, |
| | | | | | kinase beta |
| IL2RB | 22q13 | 0.0079 | IL2RB | OTHER_TARGETS | Interleukin 2 receptor, beta |
| JAK1 | 1p32.3-p31.3 | 0.0071 | JAK1 | KINASE | Janus kinase 1 (a protein tyrosine kinase) |
| PPARBP | 17q12-q21.1 | 0.0091 | PPARBP | NR_COFACTOR | PPAR binding protein |
| RIPK4 | 21q22.3 | 0.0075 | RIPK4 | KINASE | Ankyrin repeat domain 3 |
| SLC1A2 | 11p13-p12 | 0.0053 | SLC1A2 | TRANSPORTER | Solute carrier family 1 (glial high affinity glutamate transporter), |
| | | | | | member 2 |
| SRA1 | 5q31.3 | 0.0055 | APBB3 | Unclassified | FE65-like protein 2 |
| | | | SLC35A4 | TRANSPORTER | Similar to RIKEN cDNA 2610030J16 gene |
| | | | SRA1 | NR_COFACTOR | Steroid receptor RNA activator 1 |
| YES1 | 18p11.31-p11.21 | 0.0055 | YES1 | KINASE | v-yes-1 Yamaguchi sarcoma viral oncogene homolog 1 |

¹The loci in Table 6 are those with the strongest statistical evidence for disease association. The loci in Table 7 are the next best in terms of statistical evidence. These loci have a gene-based permutation p between 0.005 and 0.01 in 1,270 cases and 1,215 controls.
²Locus is a label used to assign a 1:1 relationship between a SNP and a unique part of the genome. In most instances the locus and gene are one in the same. However, in gene rich parts of the genome (where SNPs map to multiple genes), a locus may include several genes.
³In gene rich parts of the genome, some loci may have SNPs which map to several genes or have overlapping genes. Disease association may be to any of these genes.

Logistic regression analysis adjusting for the effects of Apolipoprotein E polymorphism (APOE ε4) gave 13 additional genes with significant evidence of association with AD (observed P<0.001); five of these are also reported in Tables 6 or 7. Three of these indicated significant association only in the sub-cohorts of carriers of APOE4 polymorphism, while 2 only in sub-cohorts of non-carriers of APOE4 polymorphism. Results of the logistic regression analysis are provided in Table 8.

TABLE 8
Results of stratified analysis using logistic regression analysis

| | | | | | |
|---|---|---|---|---|---|
| Locus² | Chr Location | Observed P-value | Gene³ | Target Class | Gene Description |

Observed P-value <0.001 in full data set with collection site and APOE4 status as covariates.

| | | | | | |
|---|---|---|---|---|---|
| ALK | 2p23 | 0.0006 | ALK | Kinase | Anaplastic lymphoma kinase (Ki-1) |
| C5 | 9q33-q34 | 0.0006 | C5 | 7TM Ligand | Complement component 5 |
| PAK2 | 3q29 | 0.0010 | PAK2 | Kinase | p21 (CDKN1A)-activated kinase 2 |
| CCBP2* | 3p21.3 | 0.0002 | CCBP2 | 7TM | Chemokine binding protein 2 |
| GPRC5A* | 12p13-p12.3 | 0.0003 | GPRC5A | 7TM | Retinoic acid induced 3 |
| IL2RB* | 22q13 | 0.0005 | IL2RB | Other | Interleukin 2 receptor, beta |
| MMP10_MMP1* | 11q22.3 | 0.0001 | MMP1 | Protease | Matrix metallopropeinase 1 (interstitial collagenase) |
| | | | MMP10 | Protease | Matrix metalloproteinase 10 (stromelysin 2) |
| ROR2* | 9q22 | 0.0003 | ROR2 | Kinase | Receptor tyrosine kinase-like orphan receptor 2 |

Observed P-value <0.001 in APOE4 Positive only with collection site as covariate.

| | | | | | |
|---|---|---|---|---|---|
| CHRNB4 | 15q24 | 0.0009 | CHRNB4 | Ion Channel | Cholinergic receptor, nicotinic, beta polypeptide 4 |
| KCNQ5 | 6q14 | 0.0009 | KCNQ5 | Ion Channel | Potassium voltage-gated channel, KQT-like subfamily, mmber 5 |
| PPAT | 4q12 | 0.0001 | PPAT | Protease | Phosphoribosyl pyrophosphate amidotransferase |

Observed P-value <0.001 in APOE4 Negative only with collection site as covariate.

| | | | | | |
|---|---|---|---|---|---|
| ALK^{†} | 2p23 | 0.0006 | ALK | Kinase | Anaplastic lymphoma kinase (Ki-1) |
| CLCN7 | 16p13 | 0.0005 | CLCN7 | Target Accessory | Chloride channel 7 |
| IL16 | 15q26.3 | 0.0001 | IL16 | Unclassified | Interleukin 16 (lymphocyte chemoattractant factor) |

*These genes have also been reported in the main case control analysis (see Tables 6 or 7).
^{†}This gene also gave p-value<0.001 in the full data set but NOT in the APOE4 positive subjects.

TABLE 9
Assessment Comparing All Cases to All Controls (Canadian + Cardiff)

| | Total No. | Genotypic | | Allelic | |
|---|---|---|---|---|---|
| | genotypic | Association | | Association | |
| Analysis p- | or allelic | No. tests < | Binomial | No. tests < | Binomial |
| values = p | tests | p(m) | prob ≧ m | p(m) | prob ≧ m |
| P < 0.05 | 2,930 | 144 | 0.56243 | 133 | 0.86543 |
| P < 0.01 | 2,930 | 37 | 0.06831 | 35 | 0.12640 |
| P < 0.005 | 2,930 | 21 | 0.04296 | 22 | 0.02587 |
| P < 0.001 | 2,930 | 10 | 0.00024 | 7 | 0.01042 |
| P < 0.0005 | 2,930 | 7 | 0.00014 | 6 | 0.00081 |

TABLE 10
Assessment Comparing Canadian Cases to Canadian Controls

| | Total No. | Genotypic | | Allelic | |
|---|---|---|---|---|---|
| | genotypic | Association | | Association | |
| Analysis p- | or allelic | No. tests < | Binomial | No. tests < | Binomial |
| values = p | tests | p(m) | prob ≧ m | p(m) | prob ≧ m |
| P < 0.05 | 2,889 | 117 | 0.99089 | 86 | 1.0000 |
| P < 0.01 | 2,889 | 20 | 0.94764 | 17 | 0.98823 |
| P < 0.005 | 2,889 | 11 | 0.77636 | 8 | 0.95066 |
| P < 0.001 | 2,889 | 4 | 0.16635 | 2 | 0.55159 |
| P < 0.0005 | 2,889 | 2 | 0.17732 | 2 | 0.17732 |

TABLE 11
Assessment Comparing Cardiff Cases to Cardiff Controls

| | Total No. | Genotypic | | Allelic | |
|---|---|---|---|---|---|
| | genotypic | Association | | Association | |
| Analysis p- | or allelic | No. tests < | Binomial | No. tests < | Binomial |
| values = p | tests | p(m) | prob ≧ m | p(m) | prob ≧ m |
| P < 0.05 | 2,680 | 144 | 0.17551 | 135 | 0.44196 |
| P < 0.01 | 2,680 | 33 | 0.09981 | 31 | 0.17911 |
| P < 0.005 | 2,680 | 17 | 0.13231 | 17 | 0.13231 |
| P < 0.001 | 2,680 | 5 | 0.05505 | 6 | 0.01980 |
| P < 0.0005 | 2,680 | 4 | 0.01199 | 3 | 0.04715 |

Conclusion from Population Stratification Assessment: The number of tests significant across various thresholds was not consistently inflated beyond what is expected by chance for any of the three contrasts between cases and controls (Tables 9, 10 and 11). Thus there is no evidence of population stratification between cases and controls.

### REFERENCES

Bergem A L, Engedal K, Kringlen E. The role of heredity in late-onset Alzheimer disease and vascular dementia. A twin study. Arch Gen Psychiatry 1997; 54:264-270.
Corder E H, Saunders A M, Strittmatter W J et al. Gene dose of apolipoprotein E type 4 allele and the risk of Alzheimer's disease in late onset families. Science 1993; 261:921-923.
Diagnostic and Statistical Manual of Mental Disorders DSM-IV, 4 Ed. American Psychiatric Association, 1994.
Fillenbaum G. G. Multidimensional functional assessment of older adults: the Duke Older Americans Resources and Services procedures. Hillsdale, N.J.: Lawrence Erlbaum Associates, 1988.
Fleiss J, Levin B., Paik M C. (2003) Statistical Methods for Rates and Proportions. 3^{rd} Edition. Wiley.
Folstein M F, Folstein S E, McHugh P R. “Mini-mental state”. A practical method for grading the cognitive state of patients for the clinician. J Psychiatr Res 1975; 12:189-198.
Gatz M, Pedersen N L, Berg S et al. Heritability for Alzheimer's disease: the study of dementia in Swedish twins. J Gerontol A Biol Sci Med Sci 1997; 52:M117-M125.
Gatz M, Reynolds C A, Fratiglioni L et al. Role of Genes and Environments for Explaining Alzheimer Disease. Arch Gen Psychiatry 2006; 63:168-174.
Mehta, C. and Patel, N. (1983) A Network Algorithm for Performing Fisher's Exact Test in rXc contingency tables. Journal of the American Statistical Association 78:427-434.
McKhann G, Drachman D, Folstein M, Katzman R, Price D, Stadlan E M. Clinical diagnosis of Alzheimer's disease: report of the NINCDS-ADRDA Work Group under the auspices of Department of Health and Human Services Task Force on Alzheimer's Disease. Neurology 1984; 34:939-944.
Mattis S. Mental Status Examination for organic mental syndrome in the elderly patient. In: Bellak T, Karasu T B, eds. Geriatric Psychiatry. New York: Grune et Stratton, 1976, 77-121.
Meng, Z. et al. (2003) Selection of Genetic Markers for Association Analyses, Using Linkage Disequilbrium and Haplotypes. American Journal of Human Genetics 71(1): 115-130.
Reisberg B, Ferris S H, de Leon M J, Crook T. The Global Deterioration Scale for assessment of primary degenerative dementia. Am J Psychiatry 1982; 139:1136-1139.
Roses A D., Burns D K., Chissoe S., Middleton L., St Jean P., (2005) Disease-specific target selection: A Critical First Step Down the Right Road. Drug Discovery Today 10: 177-189.
Saunders A M, Strittmatter W J, Schmechel D et al. Association of apolipoprotein E allele epsilon 4 with late-onset familial and sporadic Alzheimer's disease. Neurology 1993; 43:1467-1472.
Schmidt R, Freidl W, Fazekas F et al. The Mattis Dementia Rating Scale: normative data from 1,001 healthy volunteers. Neurology 1994; 44:964-966.
Taylor J D., Briley D., Nguyen Q., Long K., Tannone M A., Li M S., Ye F., Afshari A., Lai E., Wagner M., Chen J., Weiner M P. (2001) Flow cytometric platform for high-throughput single nucleotide polymorphism analysis. [Journal Article] Biotechniques. 30(3):661-6, 668-9, Mar.
Weir, B S. (1996) Genetic Data Analysis II. Sinauer Associates, Inc., Sunderland, Mass., pp. 109-110.
Zaykin D V, Zhivotovsky L A, Weir B S (1995) Exact tests for association between alleles at arbitrary numbers of loci. Genetica 96:169-178.

That which is claimed is:

1. A method of screening a small molecule compound for use in treating Alzheimers Disease, comprising screening a test compound against a target selected from the group consisting of the gene products encoded by ACHE, ARS2, APOC1, APOE, TOMM40, CCBP2, CCDC21, CCL26, CSNK2A1, GPRC5A, MMP10, MMP1, C190RF22, CFD, ELA2, PRTN3, THRAP5, ROR2, SCAMP5, SCG2, SLC25A4, TXNDC14, ABCD2, BUB1, CCL11, CPB2, FGR, IKBKB, IL2RB, JAK1, PPARBP, RIPK4, SLC1A2, APBB3, SLC35A4, SRA1, YES1, ALK, C5, CHRNB4, CLCN7, IL16, KCNQ5, PAK2, and PPAT, where activity against said target indicates the test compound has potential use in treating Alzheimers Disease.
